(19) United States
(12) Patent Application Publication
(10) Pub. No.: US 2026/0315284 A1
(43) Pub. Date: Oct. 15, 2026

(54) EXPANDABLE WOUND CARE APPARATUS

(71) Applicant: Aiden James Wu, Houston, TX (US)

(72) Inventor: Aiden James Wu, Houston, TX (US)

(21) Appl. No.: 19/548,738

(22) Filed: Feb. 24, 2026

Related U.S. Application Data
(60) Provisional application No. 63/765,711, filed on Mar. 2, 2025.

(51) Int. Cl. A61F 13/01; A61F 13/00

(52) U.S. Cl. CPC A61F 13/01042; A61F 2013/00093; A61F 2013/00221; A61F 2013/0028; A61F 2013/00285

(57) ABSTRACT

A system and method for an expandable wound care apparatus of expansion in a wound cavity to reduce blood loss by mechanically creating pressure to restrict blood vessels and hemostatic agent enhancing coagulation. The apparatus includes a compressed, porous, fluid-expandable body formed of a biocompatible material and dimensioned for insertion into a wound cavity, the expandable body being configured to absorb blood or bodily fluid upon contact. The apparatus may also include expand volumetrically in response to said absorption. The apparatus may also include generate outward radial compressive force against tissue within the wound cavity during expansion. The apparatus may also include at least one hemostatic agent operatively associated with the expandable body and positioned to contact blood at the wound site. The expansion of the expandable body is at rate sufficient to generates mechanical absorbent apparatus within the wound cavity while the hemostatic agent accelerates coagulation, such that mechanical compression and chemical coagulation cooperatively reduce hemorrhage.

### CROSS-REFERENCE TO RELATED APPLICATION AND PRIORITY CLAIM

This application claims priority under 35 U.S.C. § 119(e) to U.S. Provisional Patent Application No. 63/765,711 filed on Mar. 2, 2025, which is hereby incorporated by reference in its entirety.

### TECHNICAL FIELD

This disclosure relates generally to medical devices and processes. More specifically, this disclosure relates to a system and method for an expandable wound care apparatus.

### BACKGROUND

Wound care apparatuses should provide mechanical pressure and chemically induce blood coagulation to enhance hemostatic effectiveness. For example, a person impacted by a serious wound requires immediate pressure and blood coagulation to stop or slow down the bleeding. Application of pressure helps to mechanically compress the damaged blood vessels while coagulation stops or slows the bleeding to prevent significant blood loss. Without immediate compression and coagulation, significant blood loss can lead to dangerous blood pressure drop and can potentially result in life-threatening complications such as shock. The expandable wound care apparatus expands, tailoring its size to the size of the wound. The expanded wound care apparatus expands at a rate faster than its absorption rate, providing quick compression to blood and enabling the blood vessels to constrict to prevent additional blood loss. A hemostatic chemical agent embedded in the wound care apparatus expedites coagulation to help slow or stop the bleeding. The expandable wound care apparatus is essential to stabilize the injured individual's body and enhance hemostatic effectiveness until the individual is able to receive professional assistance from a healthcare professional.

### SUMMARY

This disclosure provides a system and method for an expandable wound care apparatus.

In some examples, an apparatus is provided. The apparatus includes a compressed, porous, fluid-expandable body formed of a biocompatible material and dimensioned for insertion into a wound cavity, the expandable body being configured to absorb blood or bodily fluid upon contact. The apparatus may also include expand volumetrically in response to said absorption. The apparatus may also include generate outward radial compressive force against tissue within the wound cavity during expansion. The apparatus may also include at least one hemostatic agent operatively associated with the expandable body and positioned to contact blood at the wound site. The expansion of the expandable body is at rate sufficient to generates mechanical absorbent apparatus within the wound cavity while the hemostatic agent accelerates coagulation, such that mechanical compression and chemical coagulation cooperatively reduce hemorrhage.

In other examples, an apparatus is provided. The apparatus includes a compressed, porous, fluid-expandable body formed of a biocompatible material and dimensioned for insertion into a wound cavity, the expandable body configured to absorb blood or bodily fluid upon contact therewith. The apparatus may also include expand volumetrically in response to said absorption. The apparatus may also generate outward radial compressive force against tissue within the wound cavity during expansion. The apparatus may also include at least one hemostatic agent operatively associated with the expandable body and positioned to contact blood at the wound site. The apparatus may also include wherein expansion of the expandable body is at rate sufficient to generates mechanical absorbent apparatus within the wound cavity while the hemostatic agent accelerates coagulation, such that mechanical compression and chemical coagulation cooperatively reduce hemorrhage.

Any single one or any combination of the following features may be used with the above embodiment. The apparatus may also include the outward radial pressure is maintained for at least five minutes after expansion. The apparatus may also be configured to generate an outward radial compressive force against surrounding tissue of about 5 mmHg and about 250 mmHg with contact. The apparatus may also include expansion occurs prior to complete fluid saturation of the expandable body. The apparatus may also include the expandable body comprises a plurality of expansion-directing grooves configured to guide outward radial expansion and reduce inward buckling during expansion. The apparatus may also include grooves partially segmenting the expandable body while maintaining structural continuity. The apparatus may also include a flexible tensile substrate surrounding at least a portion of the expandable body.

In still other examples, a method is provided of expansion in a wound cavity to reduce blood loss by mechanically creating pressure to restrict blood vessels and hemostatic agent enhancing coagulation. The method includes absorbing blood or bodily fluid upon contact using an expandable body, the expandable body comprising a compressed, porous, fluid-expandable body formed of a biocompatible material and dimensioned for insertion into a wound cavity. The method also includes expanding the expandable body volumetrically in response to absorption, wherein expansion of the expandable body is at rate sufficient to generates mechanical absorbent apparatus within the wound cavity while the hemostatic agent accelerates coagulation, such that mechanical compression and chemical coagulation cooperatively reduce hemorrhage. The method may also include generating outward radial compressive force against tissue within the wound cavity during expansion. Additionally, at least one hemostatic agent may be operatively associated with the expandable body and positioned to contact blood at the wound site.

Any single one or any combination of the following features may be used with the above embodiment.

Other technical features may be readily apparent to one skilled in the art from the following figures, descriptions, and claims.

### BRIEF DESCRIPTION OF THE DRAWINGS

For a more complete understanding of this disclosure, reference is made to the following description, taken in conjunction with the accompanying drawings, in which:

FIG. 1 illustrates an example an expandable wound care apparatus expandable wound care in accordance with this disclosure;

FIG. 2 illustrates an example an expandable wound care apparatus expandable wound care in accordance with this disclosure;

FIG. 3 illustrates an example expandable wound care apparatus expandable wound care in accordance with this disclosure;

FIG. 4 illustrates an example expandable wound care apparatus expandable wound care in accordance with this disclosure;

FIG. 5 illustrates an example expandable wound care apparatus expandable wound care in accordance with this disclosure;

FIG. 6 illustrates an example expandable wound care apparatus expandable wound care in accordance with this disclosure; and

FIG. 7 illustrates an example method for an expandable wound care apparatus according to this disclosure.

### DETAILED DESCRIPTION

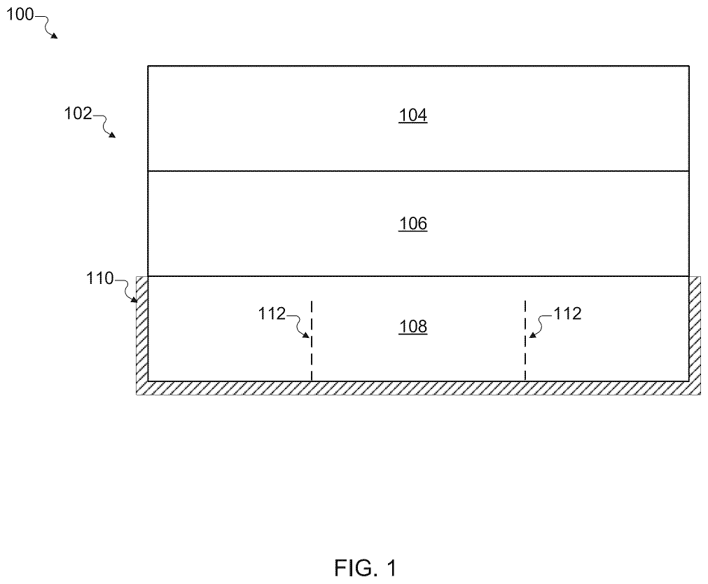

FIGS. 1 through 7, described below, and the various embodiments used to describe the principles of the present disclosure are by way of illustration only and should not be construed in any way to limit the scope of this disclosure. Those skilled in the art will understand that the principles of the present disclosure may be implemented in any type of suitably arranged device or system.

Wound care apparatuses should provide mechanical pressure and chemically induce blood coagulation to enhance hemostatic effectiveness. For example, a person impacted by a serious wound requires immediate pressure and blood coagulation to stop or slow down the bleeding. Application of pressure helps to mechanically compress the damaged blood vessels while coagulation stops or slows the bleeding to prevent significant blood loss. Without immediate compression and coagulation, significant blood loss can lead to dangerous blood pressure drop and can potentially result in life-threatening complications such as shock. The expandable wound care apparatus expands, tailoring its size to the size of the wound. The expanded wound care apparatus expands at a rate faster than its absorption rate, providing quick compression to blood and enabling the blood vessels to constrict to prevent additional blood loss. A hemostatic chemical agent embedded in the wound care apparatus expedites coagulation to help slow or stop the bleeding. The expandable wound care apparatus is essential to stabilize the injured individual's body and enhance hemostatic effectiveness until the individual is able to receive professional assistance from a healthcare professional.

FIG. 1 illustrates an example expandable wound care apparatus 100 in accordance with this disclosure. As shown in FIG. 1, the expandable wound apparatus 100 includes an expandable body 102. The expandable body 102 may include multiple layers, including a substrate layer 104, an adhesive layer 106, an expanding layer 108, and a hemostatic coating layer 110. The expanding layer 108 may also include one or more grooves 112.

The substrate layer 104 layer is a sterile, permeable embodiment serving as the structural support and framework of the expandable wound apparatus 100. Such substrate layer 104 layer may be made of gauze, rayon, non-woven or other sterile, permeable material to increase tensile strength. The adhesive layer 106 may include a biocompatible material that holds the substrate layer 104 and the expandable layer 108 together. The expandable wound care apparatus 100 contains an expandable layer 108 with channel grooves 112 to improve flexibility. The channel grooves 112 are configured to allow expansion of the expandable wound care apparatus 100 in a controlled outward manner upon activation, conforming to the wound cavity. The grooves 112 partially divide the expandable porous element while maintaining structural continuity, thereby reducing inward buckling or folding during expansion. This guided expansion improves conformity to irregular wound geometries and promotes uniform pressure distribution against surrounding tissue and damaged blood vessels.

In its in inactive state, the expandable layer 108 is compressed and durable. In its active state, fluids are absorbed by the porous, expandable layer 108, which rapidly expands to conform to the size of the wound cavity.

In various embodiments, the porous element of the expandable layer 108 may be formed from cellulose, polymeric foam, hydrogel materials, collagen-based substrate layer layers 104, synthetic sponges, or other biocompatible materials capable of absorbing fluid and expanding in volume when exposed to blood or bodily fluids. The selection of material may be varied based on desired expansion rate, absorbency, mechanical strength, and biocompatibility. The expandable layer 108 may also comprise multiple layers.

The adhesive layer 106 binder may include gelatin, collagen, polymeric adhesive layers 106, bioresorbable adhesive layers 106, or other biocompatible binding agents capable of securing layers of the expandable wound care apparatus 100 while maintaining permeability to fluids.

The hemostatic coating layer 110 may include kaolin, chitosan, zeolite, cellulose-based clotting agents, thrombin-containing compounds, or other substances capable of promoting coagulation.

In one embodiment, the expandable porous element is fully encased within the substrate layer 104. In another embodiment, the porous element is partially exposed to allow more rapid fluid contact and expansion.

In some embodiments, the hemostatic coating layer 110 is infused throughout the expandable layer 108, while in other embodiments the hemostatic coating layer 110 is coated on an external surface of the expandable wound care apparatus 100.

Although shown the expandable wound care apparatus 100 is shown as a single expandable unit. In other embodiments, multiple expandable units may be sequentially inserted into a wound cavity to accommodate deeper or irregular wound tracts.

Additionally or alternatively, the expandable wound care apparatus 100 may be pre-compressed and packaged in a sterile configuration. Additionally or alternatively, the expandable wound apparatus 100 may be partially pre-expanded prior to application as desired.

Although FIG. 1 illustrates an expandable wound apparatus 100 expandable wound care, various changes may be made to FIG. 1. For example, various components in FIG. 1 may be combined, further subdivided, replicated, omitted, or rearranged and additional components may be added according to particular needs.

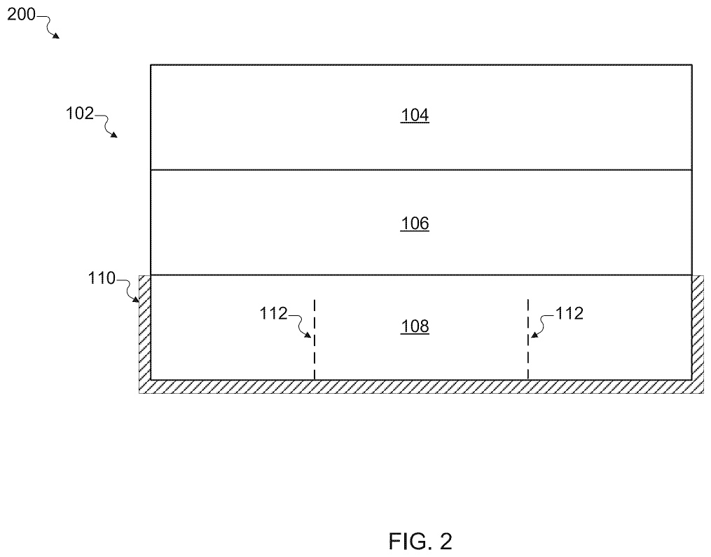

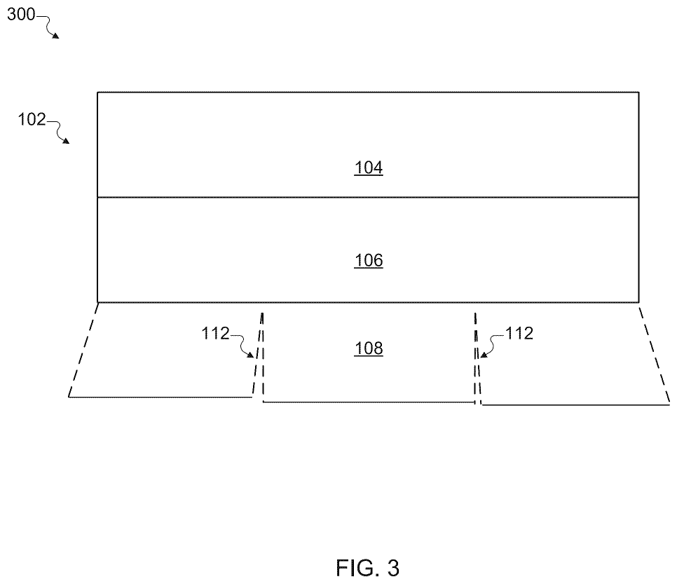

FIG. 2 illustrates an expandable wound care apparatus 200 supporting expandable wound care in accordance with this disclosure. The expandable wound care apparatus 200 is configured similarly to the expandable wound care apparatus 100 of FIG. 1, except as otherwise described. FIG. 3 illustrates an example expandable wound care apparatus 300 in accordance with this disclosure. The expandable wound apparatus 200 is configured similarly to the expandable wound apparatus 100-200 of FIG. 1-2, except as otherwise described.

As shown in FIG. 2, the expandable wound care apparatus 200 may be in an inactive or unexpanded state (such as by not contacting an activating fluid, including blood in a wound). The grooves 112 may perforate into the expanding layer 108 from a first side of the expanding layer 108, such as an outer surface of the expanding layer 108 configured to make contact with fluid in a compressed space (such as fluid or blood in a wound).

As shown in FIG. 3, the expandable wound a care apparatus 300 includes the grooves 112 in an active or expanded state (such as by contacting the expanding layer expanding layer 108, the hemostatic layer hemostatic layer 110, the grooves 112, or a combination thereof, to a fluid such as blood in a wound). In an active or expanded state, the expanding body 300 may exert force, such as a compressive force, through the expanding layer expanding layer 108.

Although FIGS. 2-3 illustrates an example expandable wound apparatus 100 expandable wound care in active and inactive stages, various changes may be made to FIG. 2. For example, various components in FIGS. 2-3 may be combined, further subdivided, replicated, omitted, or rearranged and additional components may be added according to particular needs.

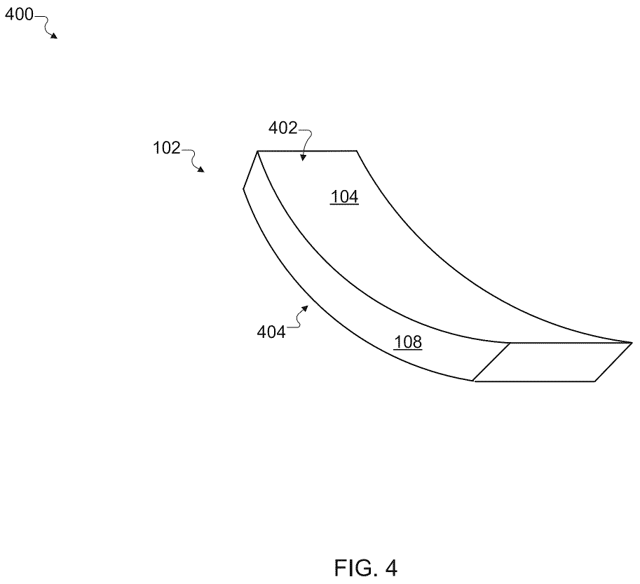

FIG. 4 illustrates an example expandable wound care apparatus 400 in accordance with this disclosure. The expandable wound care apparatus 400 is configured similarly to the expandable wound care apparatus 100-300 of FIGS. 1-3, except as otherwise described.

As shown in FIG. 4, the expandable wound care apparatus 400 is a perspective, top view of the expandable wound care apparatus 400. In particular, the expandable wound apparatus 400 illustrates a potential positioning of the substrate layer substrate layer 104 in relation to the expandable layer expanding layer 108. The expandable wound apparatus 400, in such a configuration, includes an inner surface 402 adjacent to an outer surface of the substrate layer expandable body 102 and an outer surface 404 adjacent to an outer surface of the expanding layer expanding layer 108, the outer surface 404 opposite the inner surface 402 such that the outer surface 404 is configured to expand, for example, at a faster rate than the inner surface 402 to apply a compressive force outward (such as a radially outward in response to contact with a fluid in a wound).

Although FIG. 4 illustrates example expandable wound apparatus 400 expandable wound care, various changes may be made to FIG. 4. For example, various components in FIG. 4 may be combined, further subdivided, replicated, omitted, or rearranged and additional components may be added according to particular needs.

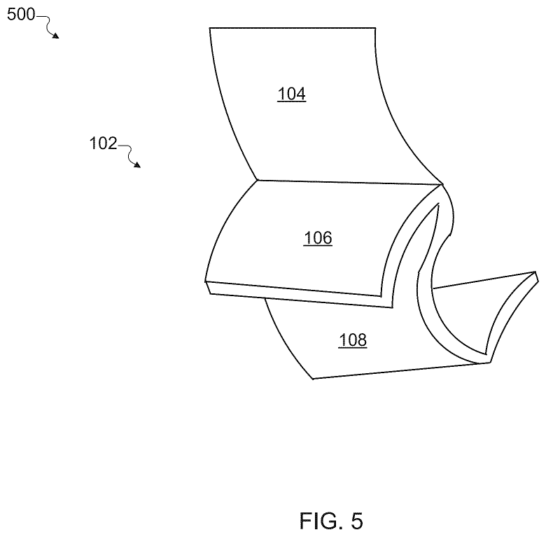

FIG. 5 illustrates an example expandable wound apparatus 500 expandable wound care in accordance with this disclosure. The expandable wound apparatus 500 is configured similarly to the expandable wound apparatus 100-400 of FIGS. 1-4, except as otherwise described.

As shown in FIG. 5, the expandable wound care apparatus 500 illustrates the positioning of the adhesive layer adhesive layer 106, such as between the substrate layer substrate layer 104 and the expanding layer expanding layer 108. However, the adhesive layer adhesive layer 106 may be in any desired position and may include any desired combination of layers, including only one, sufficient to adhere the substrate layer expanding layer 108 to the expanding layer hemostatic coating layer 110 (FIG. 1). Additionally or alternatively, the adhesive layer expanding layer 108 may also be configured to adhere to the hemostatic layer hemostatic layer hemostatic coating layer 110, individual portions of the expanding layer hemostatic coating layer 110 (such as between the grooves 112), or a combination thereof.

Although FIG. 5 illustrates an example expandable wound apparatus 500 expandable wound care, various changes may be made to FIG. 5. For example, various components in FIG. 5 may be combined, further subdivided, replicated, omitted, or rearranged and additional components may be added according to particular needs.

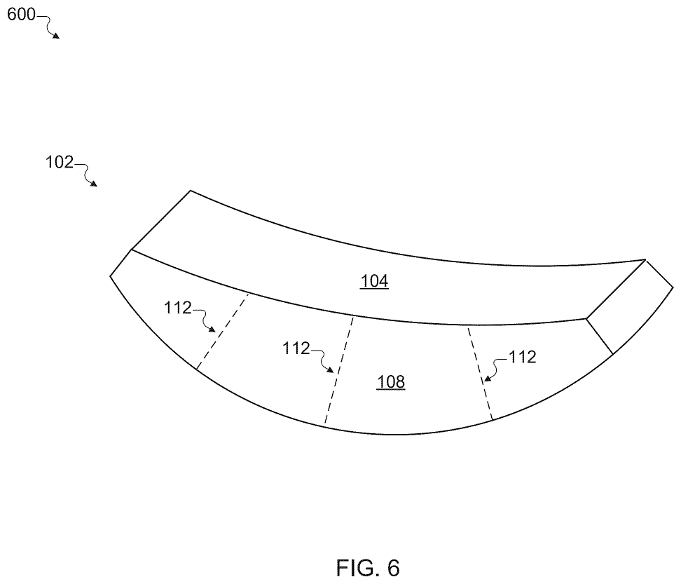

FIG. 6 illustrates an example expandable wound apparatus 600 supporting expandable wound care in accordance with this disclosure.

As shown in FIG. 6, the expandable wound care apparatus 600 includes the expanding layer expanding layer 108 with the grooves 112 in an expanded state such that that outer surface of the expanding layer expanding layer 108 is larger than an inner surface opposite the outer surface to provide a compressive force outward (such as toward the outer surface) in response to contact with a fluid (such as blood in a wound). As described above, the expanding layer expanding layer 108 may also include the hemostatic layer hemostatic layer hemostatic coating layer 110 to apply further, desired pressure or otherwise hemostatic properties in addition to the applied compressive force of the expanding layer expanding layer 108.

Although FIG. 6 illustrates an example expandable wound apparatus 600 expandable wound care, various changes may be made to FIG. 6. For example, various components in FIG. 6 may be combined, further subdivided, replicated, omitted, or rearranged and additional components may be added according to particular needs.

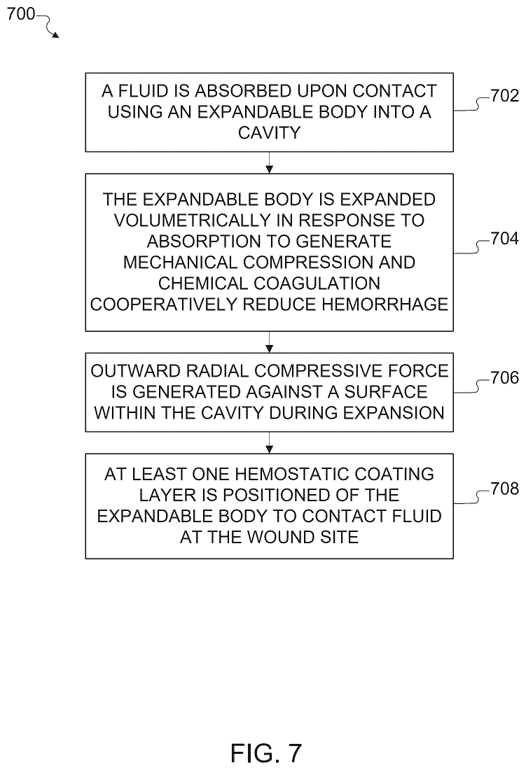

FIG. 7 illustrates an example method 700 for expandable wound care according to this disclosure. For ease of explanation, the method 700 of FIG. 7 is described as being performed using the expandable wound care apparatus 100 of FIGS. 1-6. However, the method 700 may be used with any other suitable system and any other suitable expandable wound apparatus supporting expandable wound care.

As shown in FIG. 7, a fluid is absorbed upon contact using an expandable body into a cavity at step 702. For example, the expandable wound apparatus 100 may be inserted into a cavity (such as a would cavity that may include one or more fluids, including blood).

The expandable body is expanded volumetrically in response to absorption, wherein expansion of the expandable body is at rate sufficient to generates mechanical absorbent apparatus within the wound cavity while the hemostatic agent accelerates coagulation, such that mechanical compression and chemical coagulation cooperatively reduce hemorrhage at step 704. For example, the expandable wound care apparatus 100 may include an expanding layer 108 that is configured to expand upon contact with the fluid (such as blood).

Outward radial compressive force is generated against a surface within the cavity during expansion at step 706. For example, upon contacting a fluid (such as blood in wound), the expanding layer 108 generates a radial compressive force outward toward a surface of the cavity (such as a wound). To do so, the expanding layer 108 may include one or more grooves 112 multiple of the expandable wound care apparatus 100, or a combination thereof.

At least one hemostatic coating layer is positioned of the expandable body to contact fluid at the wound site at step 708. For example, the expandable wound care apparatus 100 may include a, a hemostatic layer hemostatic layer hemostatic coating layer 110 to contact the fluid within a cavity (such as blood in a wound) to apply additional pressure, provide desired hemostatic effects, or other desired qualities.

Although FIG. 7 illustrates one example of a method 700 for expandable wound care, various changes may be made to FIG. 7. For example, while shown as a series of steps, various steps in FIG. 7 may overlap, occur in parallel, or occur any number of times.

It may be advantageous to set forth definitions of certain words and phrases used throughout this patent document. The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in the present application should not be read as implying that any particular element, step, or function is an essential or critical element that must be included in the claim scope. The scope of patented subject matter is defined only by the allowed claims. Moreover, none of the claims invokes 35 U.S.C. § grooves 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” or “controller” within a claim is understood and intended to refer to structures known to those skilled in the relevant art, as further modified or enhanced by the features of the claims themselves, and is not intended to invoke 35 U.S.C. § grooves 112(f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An apparatus comprising:
an expandable body comprising a biocompatible material and dimensioned for insertion into a cavity, the expandable body being configured to:
absorb blood or bodily fluid upon contact;
expand volumetrically in response to absorption; and
generate outward radial compressive force against tissue within the cavity during expansion;
at least one hemostatic agent operatively associated with the expandable body and positioned to contact blood at the cavity; and
wherein expansion of the expandable body is at rate sufficient to generate mechanical absorbent apparatus within the cavity while the hemostatic agent accelerates coagulation, such that mechanical compression and chemical coagulation cooperatively reduce hemorrhage within the cavity.

2. The apparatus of claim 1, wherein outward radial pressure is maintained for at least five minutes after expansion.

3. The apparatus of claim 1, wherein the expandable body generates an outward radial compressive force against surrounding tissue of about 5 mmHg and about 250 mmHg with contact.

4. The apparatus of claim 1, wherein expansion occurs prior to complete fluid saturation of the expandable body.

5. The apparatus of claim 1, wherein the expandable body comprises a plurality of grooves configured to guide outward radial expansion and reduce inward buckling during expansion.

6. The apparatus of claim 5, wherein the grooves partially segment the expandable body while maintaining structural continuity.

7. The apparatus of claim 1, further comprising a flexible tensile substrate surrounding at least a portion of the expandable body.

8. An apparatus comprising:
an expandable body comprising a biocompatible material dimensioned for insertion into a wound cavity, the expandable body configured to:
absorb blood or bodily fluid upon contact therewith;
expand volumetrically in response to absorption; and
generate outward radial compressive force against tissue within the wound cavity during expansion; and
at least one hemostatic agent operatively associated with the expandable body and positioned to contact blood or bodily fluid at the wound cavity; and
wherein expansion of the expandable body is at rate sufficient to generate mechanical absorbent apparatus within the wound cavity while the hemostatic agent accelerates coagulation, such that mechanical compression and chemical coagulation cooperatively reduce hemorrhaging in the wound cavity.

9. The apparatus of claim 8, wherein outward radial pressure is maintained for at least five minutes after expansion.

10. The apparatus of claim 8, wherein the expandable body generates an outward radial compressive force against surrounding tissue of about 5 mmHg and about 250 mmHg with contact.

11. The apparatus of claim 8, wherein expansion occurs prior to complete fluid saturation of the expandable body.

12. The apparatus of claim 8, wherein the expandable body comprises a plurality of grooves are configured to guide outward radial expansion and reduce inward buckling during expansion.

13. The apparatus of claim 12, wherein the grooves partially segment the expandable body while maintaining structural continuity.

14. The apparatus of claim 8, further comprising a flexible tensile substrate surrounding at least a portion of the expandable body.

15. A method of reducing blood loss in a wound cavity by mechanically creating pressure to restrict blood vessels and providing a hemostatic agent enhancing coagulation, comprising:
absorbing blood or bodily fluid upon contact with an expandable body, the expandable body comprising a compressed, porous, fluid-expandable body formed of a biocompatible material and dimensioned for insertion into the wound cavity;
expanding the expandable body volumetrically in response to absorption, wherein expansion of the expandable body is at rate sufficient to generate a mechanical absorbent apparatus within the wound cavity while the hemostatic agent accelerates coagulation, such that mechanical compression and chemical coagulation cooperatively reduce hemorrhage;
generating outward radial compressive force against tissue within the wound cavity during expansion using the expandable body; and
positioning at least one hemostatic coating layer of the expandable body to contact fluid at the wound cavity.

16. The method of claim 15 further comprising the step of locating the hemostatic agent on an external surface of the expandable body.

17. The method of claim 15 further comprising fully enclosing the expandable body within a substrate.

18. The method of claim 15, wherein expanding the expandable body volumetrically in response to absorption further comprises maintaining compressive force of the expandable body under movement or transport.

19. The method of claim 15, wherein expanding the expandable body volumetrically in response to absorption further comprises partially exposing the expandable body to allow fluid contact.

20. The method of claim 15 further comprising sequentially inserting multiple expandable bodies into the wound cavity to increase cumulative absorption force.
